(19) United States
(12) Patent Application Publication
(10) Pub. No.: US 2017/0172731 A1
(43) Pub. Date: Jun. 22, 2017

(54) Biocompatible electro-optics package for in vivo use

(71) Applicant: Novartis AG, Basel (CH)

(72) Inventors: Michael F. Mattes, Arlington, TX (US); Mark A. Zielke, Fort Worth, TX (US); Jonathan McCann, Mansfield, TX (US); Richard D. Lintern, Cambridgeshire (GB); Samuel Pollock, Hertfordshire (GB)

(21) Appl. No.: 14/976,070

(22) Filed: Dec. 21, 2015

(51) Int. Cl. A61F 2/16

(52) U.S. Cl. CPC A61F 2/1624; A61F 2/1691; A61F 2002/1689

(57) ABSTRACT

A bio-compatible packaging for an optoelectronic device is presented, to essentially eliminate moisture ingress and corrosion of the internal electronics of the device after it is implanted for in-vivo use. In some implementations, the optoelectronic device includes an optoelectronic assembly that includes an electronic module, an optoelectronic module, a power source, configured to energize the electronic module and the optoelectronic module, and an electronic interconnect to provide electronic couplings between the electronic module, the optoelectronic module, and the power source. The device further includes a bio-compatible packaging, having a transparent front window and a transparent back window, the bio-compatible packaging configured to enable light to enter the optoelectronic device through the front window, propagate through the optoelectronic module, and leave the optoelectronic device through the back window, and to hermetically seal the optoelectronic assembly.

### TECHNICAL FIELD

This patent document is related to electronic and optoelectronic devices. In more detail, this patent document is related to partially transparent optoelectronic devices that include a hermetic bio-compatible packaging for in vivo use.

### BACKGROUND

Up to date, pacemakers are the prime examples of in-vivo electronic devices. The pacing leads are connected to the pacing device typically with receptacle-and-plug type connections. In these devices, non-corrosive metals, insulation, and moisture barriers are used to maintain a projected lifetime of up to 10 years. These connections are large and not hermetic. Therefore, unfavorable leakage currents can be induced during the operation of the device. These leakage currents are often mitigated through the use of insulation and distance. Leakage currents are also not as critical in pacemaker applications since the leads only carry current when the device is sending a pacing pulse.

Recently, various electro-active intraocular lens (EA-IOL) systems with several electronic modules have been proposed for ophthalmic in vivo use. The modules of these EA-IOLs are electronically coupled by electronic connections. The moisture and corrosion protection of these electronic modules and their connections require packagings that deliver highly efficient sealing. Moreover, at least a portion of these packagings needs to be transparent for the proper operation of the embedded IOL itself. However, in an EA-IOL there is no room for the large electrical connections of the pacemakers. In addition, the power supplies of these EA-IOLs must be quite small, they are continuously operated, and all electronic modules are quite close to each other. To avoid moisture ingress, followed by corrosion and leakage currents, in such systems the electronic modules and their electronic connections must be isolated very efficiently from the in vivo environment via a protective packaging.

Somewhat related interconnect schemes have been proposed in the past, such as a high-density, chip-level integrated interconnect packaging system in the article “Microelectronic Packaging for Retinal Prostheses” by D. C. Rodger and Y-C. Tai, in IEEE Engineering in Medicine and Biology Magazine, p. 52, September 2005. However, the described scheme applied a parylene polymer layer as the coating and thus is likely to suffer from moisture ingress into the packaging over years, causing leakage currents and eventually, corrosion of the internal electronics of these devices.

For at least the above reasons, hermetically sealed and bio-compatible packagings are needed for optoelectronic devices that are at least partially transparent to let light into the optoelectronic device itself. These packagings need to be small enough for implantation into an eye, and essentially eliminate moisture ingress and leakage currents by providing reliable sealing for at least 10 years even when exposed to the salinity conditions of biological tissue.

### SUMMARY

Embodiments in this patent document address the above challenges by introducing a bio-compatible packaging for an optoelectronic device to essentially eliminate moisture ingress and corrosion of the internal electronics of the device after it was implanted for in-vivo use.

In some embodiments, an optoelectronic device is comprising an optoelectronic assembly including an electronic module; an optoelectronic module; a power source, configured to energize the electronic module and the optoelectronic module; and an electronic interconnect to provide electronic couplings between the electronic module, the optoelectronic module, and the power source; and a bio-compatible packaging, having a transparent front window and a transparent back window, the bio-compatible packaging configured to enable light to enter the optoelectronic device through the front window, propagate through the optoelectronic module, and leave the optoelectronic device through the back window; and to hermetically seal the optoelectronic assembly.

### BRIEF DESCRIPTION OF THE DRAWINGS

FIG. 1 illustrates an optoelectronic assembly 100.

FIG. 2 illustrates a perspective view of an optoelectronic device 200 with a bio-compatible packaging 300.

FIG. 3 illustrates a side view of a two-layer embodiment of the optoelectronic device 200 with a bio-compatible packaging 300.

FIG. 4 illustrates a side view of a three-layer embodiment of the optoelectronic device 200 with a bio-compatible packaging 300.

FIG. 5 illustrates a side view of an optoelectronic device 200 with a bio-compatible packaging 300, embedded in a soft outer packaging 400.

FIG. 6 illustrates an embodiment of a hermetic electronic interconnect 140.

### DETAILED DESCRIPTION

Embodiments described herein address the above needs and challenges by introducing an optoelectronic device that has a bio-compatible packaging to provide hermetic sealing for the electronic modules of the optoelectronic device and their connections. Embodiments of this optoelectronic device have various advantageous aspects, including the followings.

(1) Embodiments can provide long-lifetime environmental protection for the electronic modules of the optoelectronic device and their electronic connections. Embodiments can be corrosion-proof for at least 10 years, thus enabling the implantation of this device for long term in-vivo use.

(2) Embodiments are, at least in part, optically transmissive, and thus are well-suited for housing electro-active IOLs.

(3) Embodiments are biocompatible, suitable for implantation into patients.

(4) Embodiments have a form factor sufficiently small to enable implantation of these optoelectronic devices into the capsular bag of the eye.

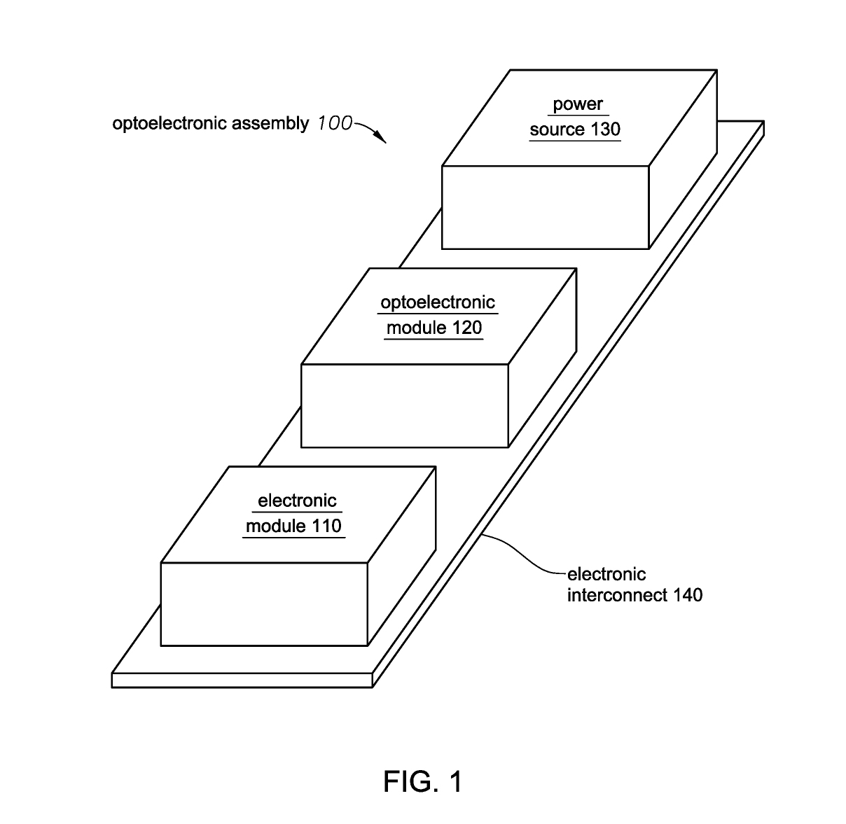

FIG. 1 illustrates an embodiment of an optoelectronic assembly 100. The optoelectronic assembly 100 can include an electronic module 110, an optoelectronic module 120, and a power source 130, to energize the electronic module 110 and the optoelectronic module 120. The optoelectronic assembly 100 can further include an electronic interconnect 140 to provide electronic couplings between the electronic module 110, the optoelectronic module 120, and the power source 130. When properly mated with the electronic module 110, the optoelectronic module 120, and the power source 130, the electronic interconnect 140 can be hermetically sealed, to prevent moisture ingress into the optoelectronic assembly 100.

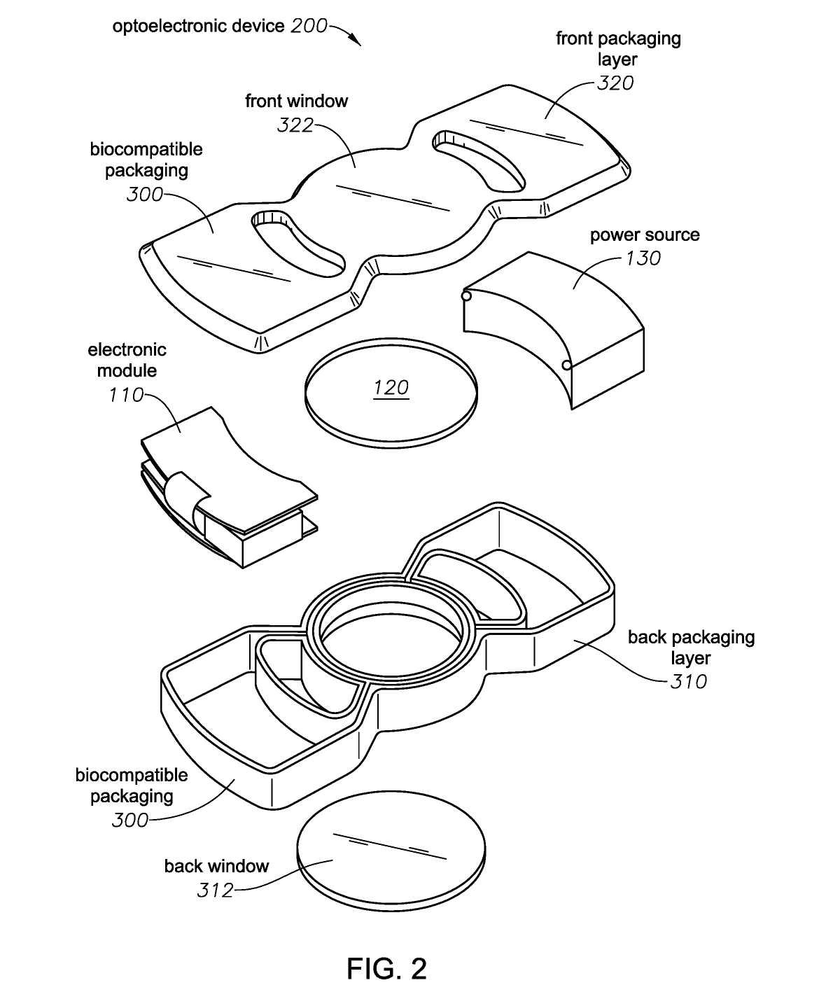

FIG. 2 illustrates an embodiment of an optoelectronic device 200 that can include the optoelectronic assembly 100 with the electronic module 110, the optoelectronic module 120, and the power source 130. The optoelectronic device 200 can further include a bio-compatible packaging 300, having a transparent back window 312, and a transparent front window 322. The bio-compatible packaging 300 can be configured to enable light to enter the optoelectronic device 200 through the front window 322, propagate through the optoelectronic module 120, and leave the device 200 through the back window 312. This is one of the aspects in which embodiments of the optoelectronic device 200 differ from pacemakers that are typically not transparent.

The bio-compatible packaging 300 can also hermetically seal the optoelectronic assembly 100 to prevent moisture ingress and corrosion of the electronic modules and their connections. Sealing is a primary functionality, given that the optoelectronic device 200 has to work in vivo after implantation for an extended period, such as 10 years or longer.

In embodiments of the optoelectronic device 200, the electronic module 110 can be an electric module, an integrated circuit, a control circuit, or an actuator. In some cases, the electronic module 110 can be a combination of more than one of these elements. The electronic module 110 can be configured to generate and to send control signals to the optoelectronic module 120. The control signals can be sent through the electronic interconnect 140.

The optoelectronic module 120 can include an electroactive Intra-Ocular Lens (EA-IOL). Such EA-IOLs can provide at least two functionalities. First, they restore vision after the removal of the natural cataractous lens of the eye. Second, their optical characteristics, including their optical power, are adjustable. Thus, EA-IOLs can actively adjust their optical characteristics, such as an optical power, in response to the control signal received from the electronic module 110. The adjustment can be performed in various ways. In some embodiments, the EA-IOL itself can have an actuator that modifies the optical power in response to the control signal. In some embodiments, the actuator can be physically separate, or located at some distance from the IOL itself, and actuate the IOL in a mechanical manner. In such embodiments, the optoelectronic module 120, or the Electro-Active IOL, can be defined to include the electronically controlled actuator, in spite of its physical separation.

In some embodiments, the optical power of the EA-IOL can be adjusted by up to 4 diopters. In other cases, the optical power can be adjusted by up to 2 diopters.

Since both the electronic module 110 and the optoelectronic module 120 need to be electronically energized, the optoelectronic device 200 can include a battery stack in the power source 130. This battery stack can provide the electrical energy needed to operate the electronic module 110 and the optoelectronic module 120, typically through the electronic interconnect 140. In other embodiments, the power source 130 can include power sources other than batteries, such as an energy harvesting device, or a fuel cell.

In the EA-IOL, and in other embodiments, the optoelectronic module 120 can be optically transmissive. Correspondingly, in some embodiments, the entire back face and front face of the optoelectronic device 200 can be transmissive, optically clear. In other embodiments, only a portion of these faces can be transmissive, or optically clear, such as the back window 312 and the front window 322 of the bio-compatible packaging 300. These aspects are part of the entire optoelectronic device 200 itself being configured to let light propagate through. In an EA-IOL implementation, the transmitted light travels from the cornea, through the pupil, and through the optoelectronic device 200, eventually to arrive to the retina of the eye.

As mentioned before, embodiments of the optoelectronic device 200 can be designed to prevent moisture ingress in vivo for 10 years, or longer, after implantation. To express this concept quantitatively, in some embodiments, the bio-compatible packaging 300 can include a packaging material with a helium permeability less than 10¹⁴ g/(cm*sec*torr) at a thickness of 100 microns over 20 years. In other embodiments, the packaging material can have a helium permeability less than 10¹⁴ g/(cm*sec*torr) at a thickness of 200 microns over 20 years.

Several materials, such as silicones, epoxies and polymers in general can be unsuitable to deliver such a sealing performance. Therefore, the packaging material of the bio-compatible packaging 300 needs to include materials that can deliver such a performance, such as sapphire, quartz, glass, transparent ceramics, and combination thereof. Some portions of the bio-compatible packaging 300 may also include metals that satisfy these criteria, including Ti, Au, Pt, or Nb and their alloys. The packaging materials employed in the bio-compatible packaging 300 in most embodiments are bio-compatible.

In ophthalmological applications, like in the case of Electro-Active Intra Ocular Lenses, embodiments of the optoelectronic device 200 can have a form factor to fit into a capsular bag of an eye, and thus be implantable into the capsular bag of the eye from where the original cataractous lens has been removed in a preceding step of a cataract surgical procedure.

Accordingly, embodiments of the optoelectronic device 200 can have a lateral extent, such as a diameter, less than 12 mm. Further, in some embodiments, a thickness of the optoelectronic device 200 can be less than 5 mm, and in others, less than 3 mm.

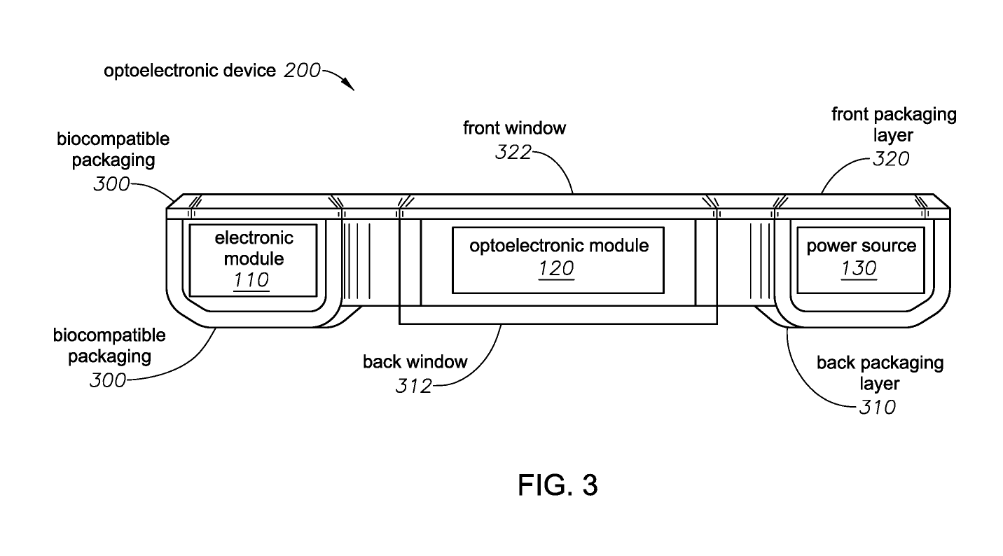

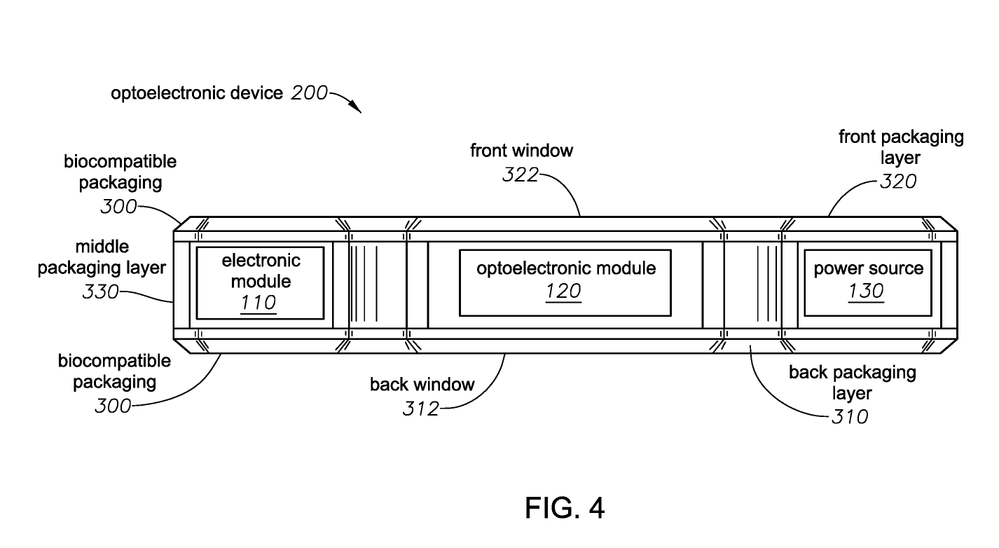

FIG. 3 and FIG. 4 illustrate two embodiments of the optoelectronic device 200 in some detail from a side view.

FIG. 3 illustrates an embodiment of the bio-compatible packaging 300 that includes a back packaging layer 310, and a front packaging layer 320, attached to the back packaging layer 310. As described earlier, each of these packaging layers can be partially optically transmissive. In the shown embodiment, the back packaging layer 310 can include the back window 312, and the front packaging layer 320 can include the front window 322 to be able to transmit light to and from the optoelectronic module 120.

The back packaging layer 310 and the front packaging layer 320 can be configured to house the optoelectronic assembly 100, and to form a hermetically sealed packaging for the optoelectronic assembly 100.

Housing the optoelectronic assembly 100 can be implemented in different ways. In some embodiments, the back and front layers 310-320 of the biocompatible packaging 300 can be configured to house the electronic module 110, the optoelectronic module 120, and the power source 130 in connected spaces, or bays, that are in fluid communication, and thus are not sealed from each other.

In other embodiments, the biocompatible packaging 300 can be configured to house the electronic module 110, the optoelectronic module 120, and the power source 130 in at least two spaces that are sealed from each other. FIG. 3 illustrates an embodiment of the optoelectronic device 200, where the bio-compatible packaging 300 is made primarily of transparent glass, and the modules 110, 120, and 130 are in separately sealed spaces, or bays.

In embodiments, the back packaging layer 310 and the front packaging layer 320 can be attached by at least one of laser-welding and metal-to-metal seals. This attaching method transfers only a low amount of heat to the modules of the optoelectronic assembly 100 during fabrication, and thus can avoid damaging the functionality of the modules during assembly.

FIG. 4 illustrates another embodiment of the optoelectronic device 200, where the bio-compatible packaging 300 includes a back packaging layer 310, a middle packaging layer 330, attached to the back packaging layer 310, and a front packaging layer 320, attached to the middle packaging layer 330. Again, at least some of the attaching can be performed by laser-welding or metal-to-metal seals.

As in the embodiment of FIG. 3, the back packaging layer 310, the middle packaging layer 330, and the front packaging layer 320 can be configured to house the optoelectronic assembly 100. Housing the optoelectronic assembly 100 can be implemented in different ways. In some embodiments, the biocompatible packaging 300 can be configured to house the electronic module 110, the optoelectronic module 120, and the power source 130 in connected spaces, or bays that are in fluid communication, and thus are not sealed from each other.

In other embodiments, the biocompatible packaging 300 can be configured to house the electronic module 110, the optoelectronic module 120, and the power source 130 in two or more spaces that are sealed from each other. FIG. 4 illustrates an embodiment of the optoelectronic device 200, where the bio-compatible packaging 300 is made primarily of transparent glass, and the electronic module 110, optoelectronic module 120, and power source 130 are in separately sealed spaces, or bays. The back, middle and front packaging layers 310-320-330 can be configured to form a hermetically sealed packaging for the optoelectronic assembly 100.

A further aspect of moisture managements can be implemented in some embodiments of the optoelectronic device 200 by including at least one of a desiccant, a getter, silica, calcium, a moisture-reducing agent, and a moisture capture material. Any one of these materials or agents can absorb or reduce the very low amount of moisture that still managed to seep through the bio-compatible packaging 300.

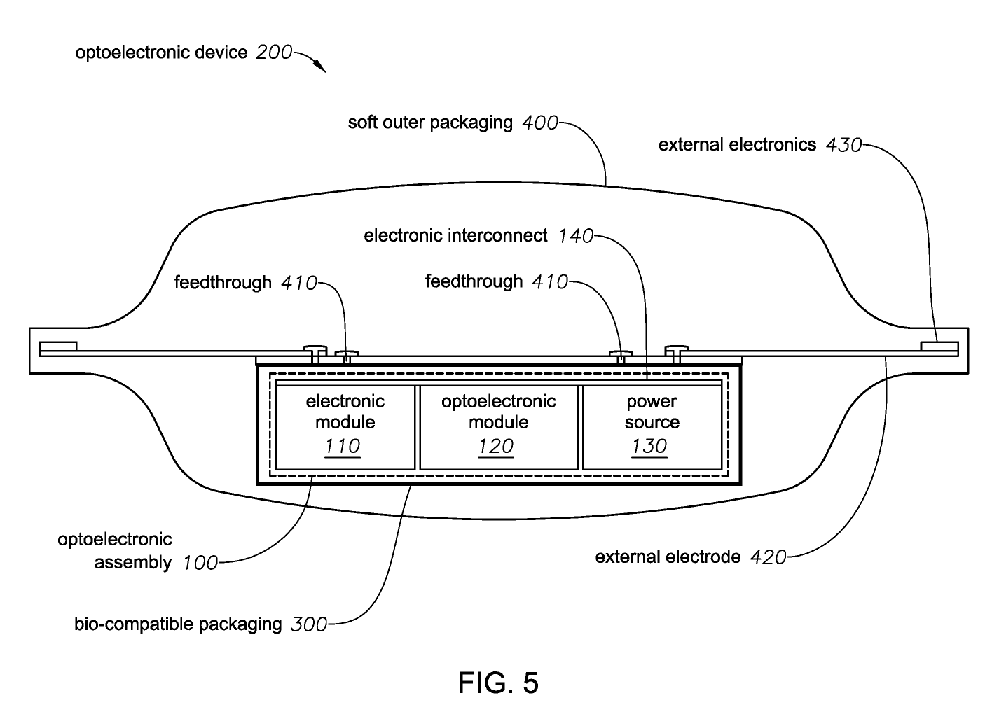

FIG. 5 illustrates that some embodiments of the optoelectronic device 200 can have somewhat sharped features or edges. These can be deleterious for the functionality of the device 200 because they can tear the surrounding tissue, for example. Therefore, some embodiments of the optoelectronic device 200 can further include a soft outer packaging 400. This soft outer packaging 400 can be configured to round the edges and sharp features of the bio-compatible packaging 300. Materials that can be useful for the formation of the embodiments of the soft outer packaging 400 can include polymer, silicone, or AcrySof, a known IOL material.

In ophthalmic implementations, where the optoelectronic module 120 is an Electro-Active IOL, the optoelectronic module 120 may be configured to provide an adjustable optical power in the range of 0-4 diopters, or 0-2 diopters, and the soft outer packaging 400 can provide an optical power in the range of 6-30 diopters. This latter optical power may not be adjustable in some embodiments.

Some embodiments of the optoelectronic device 200 can include means for electronic communication between the outside of the biocompatible packaging 300 and the optoelectronic assembly 100 inside the packaging 300. In some of these implementations, the biocompatible packaging 300 can include one or more sealed feedthroughs 410 for electronically coupling the optoelectronic assembly 100 inside the packaging 300 to an external electronics 430 through one or more external electrodes 420, positioned in the soft outer packaging 400. In these embodiments, the feedthrough 410 and the external electrode 420 can form a signal route for the external electronics 430 to signal the optoelectronic assembly 100 inside the biocompatible packaging 300. The external electronics 430 can include a sensor, a charging connector, a connector for electronic devices even farther out, or a receiver for receiving signals wirelessly.

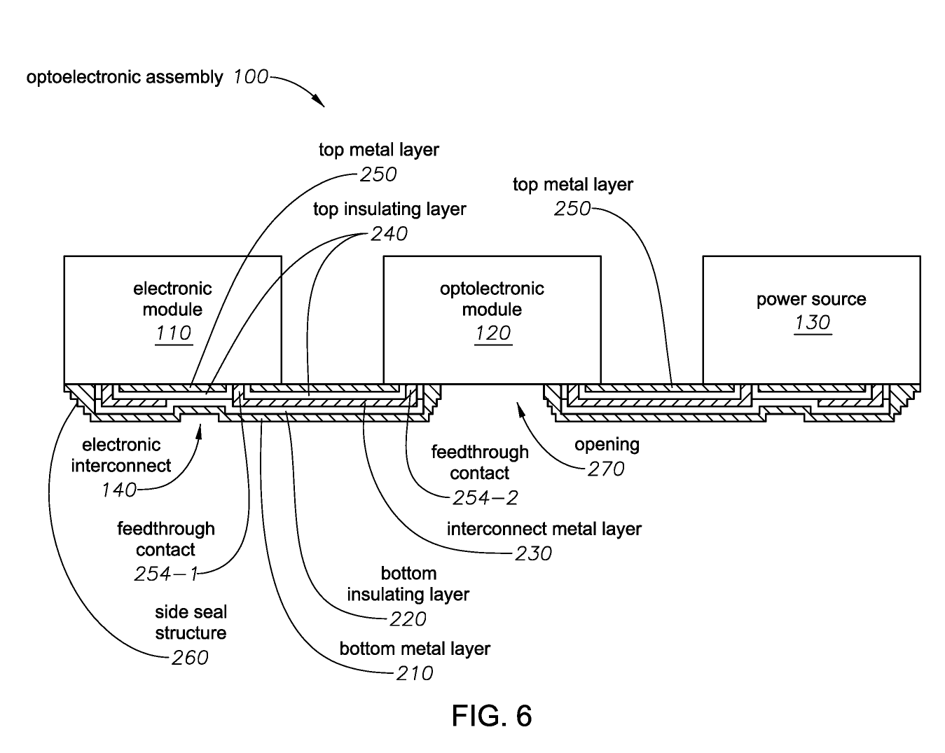

FIG. 6 shows one embodiment of the optoelectronic assembly 100 in some detail. The optoelectronic assembly 100 can include the electronic module 110, the optoelectronic module 120, and the power source 130. These can be connected by the electronic interconnect 140. The electronic interconnect 140 can be hermetic or non-hermetic, since the bio-compatible packaging 300 already provides a hermetic seal for the optoelectronic assembly 100 that substantially eliminates moisture ingress.

Nevertheless, in some embodiments, the electronic interconnect 140 can be hermetic as well. Such designs can further increase the protection of the optoelectronic assembly 100 against moisture and corrosion, extending the functional lifetime of the optoelectronic device 200. Such embodiments can include an outer seal structure, often made of metal, as metals such as Nb, Au, Pt, Ti, and their alloys, as these metals provide exceedingly low permeability over long time periods at remarkably low thicknesses.

In detail, the outer seal structure in such hermetic electronic interconnects 140 can include a bottom metal layer 210, to provide an additional base protection against the saline moisture that may seep through the biocompatible packaging 300 over time. Next, the hermetic electronic interconnect 140 can include a bottom insulating layer 220, on the bottom metal layer 210 to electronically insulate the bottom metal layer 210 from the internal electronic connections.

The hermetic electronic interconnect 140 can further include an interconnect metal layer 230 on the bottom insulating layer 220, patterned to form electrical connections between feedthrough contacts 254-1 and 254-2 that are electronically coupled to modules of the assembly 100. In FIG. 6, the feedthrough contact 254-1 is electronically coupled to the electronic module 110, and the feedthrough contact 254-2 is electronically coupled to the optoelectronic module 120.

The hermetic electronic interconnect 140 can further include a patterned top insulating layer 240 on the interconnect metal layer 230, to electronically insulate the interconnect metal layer 230. The top insulating layer 240 can be also patterned to form feedthrough holes to accommodate the feedthrough contacts 254-1 and 254-2.

The hermetic electronic interconnect 140 can finally include a top metal layer 250, on the top insulating layer 240. The bottom metal layer 210, the top metal layer 250 and a side seal structure 260 complete a hermetic seal of the electronic interconnect 140. This top metal layer 250 can be patterned to accommodate the feedthrough contacts 254-1 and 254-2. The just described hermetic electronic interconnect 140 can be electronically coupled to the electronic module 110, optoelectronic module 120, and power source 130 of the optoelectronic assembly 100 via the feedthrough contacts 254 to facilitate the energizing the modules 110 and 120 by the power source 130, and to facilitate the electronic signaling from the electronic module 110 to the optoelectronic module 120.

In FIG. 6, the electronic interconnect 140 is shown to have two separate portions, separated by an opening 270 that allows the unfettered transmission of light to and from the optoelectronic module 120. This opening/hole 270 can be implemented either by fabricating the interconnect in two separate portions, or as a single interconnect with an optically transmissive opening 270 in it, in which case the cross sectional plane of FIG. 6 cuts through the opening 270. In either case, the opening 270 of the electronic interconnect 140 can be aligned with the optoelectronic module 120, the back window 310 and the front window 320 to let the light from the front window 322 through to the optoelectronic module 120, to ensure the proper operation of the optoelectronic device 200, especially when the optoelectronic module is an Electro-Active IOL.

Such hermetic electronic interconnects 140 can be fabricated in a bottom-up or in a top-down manner. The bottom-up fabrication processes start by depositing the bottom metal layer 210 first and build the structure from there on. The top-down fabrication processes can start by depositing the top metal layer 250 on a planar face of the modules 110-120-130 and build the structure from there on. Each approach, and other variants, can have their own advantages and disadvantages.

While this specification contains many specifics, these should not be construed as limitations on the scope of the invention or of what can be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

1. An optoelectronic device, comprising:
an optoelectronic assembly including
an electronic module;
an optoelectronic module;
a power source, configured to energize the electronic module and the optoelectronic module; and
an electronic interconnect to provide electronic couplings between the electronic module, the optoelectronic module, and the power source; and
a bio-compatible packaging, having a transparent front window and a transparent back window, the bio-compatible packaging configured
to enable light to enter the optoelectronic device through the front window, propagate through the optoelectronic module, and leave the optoelectronic device through the back window; and
to hermetically seal the optoelectronic assembly.

2. The optoelectronic device of claim 1, the electronic module comprising:
at least one of an electric module, an integrated circuit, a control circuit, an actuator, and a combination thereof, configured to generate and to send control signals to the optoelectronic module.

3. The optoelectronic device of claim 2, the optoelectronic module comprising:
an electroactive Intra-Ocular Lens, configured
to receive the control signals from the electric module, and
to adjust an optical characteristics of the electroactive Intra-Ocular Lens in response to the control signal.

4. The optoelectronic device of claim 1, the power source comprising:
a battery stack.

5. The optoelectronic device of claim 1, wherein:
the optoelectronic module is optically transmissive.

6. The optoelectronic device of claim 1, wherein:
the front window and the back window of the bio-compatible packaging are optically clear.

7. The optoelectronic device of claim 1, the bio-compatible packaging comprising:
a packaging material with a helium permeability less than 10⁻¹⁴ g/(cm*sec*torr) at a thickness of 200 microns over a period of 20 years.

8. The optoelectronic device of claim 7, wherein:
the packaging material has a helium permeability less than 10⁻¹⁴ g/(cm*sec*torr) at a thickness of 100 microns over a period of 20 years.

9. The optoelectronic device of claim 7, wherein:
the packaging material is bio-compatible.

10. The optoelectronic device of claim 1, the bio-compatible packaging comprising:
a packaging material, including one of sapphire, quartz, glass, transparent ceramics, and combination thereof.

11. The optoelectronic device of claim 1, wherein:
the optoelectronic device has a form factor to fit into a capsular bag of an eye, with a lateral extent less than 12 mm.

12. The optoelectronic device of claim 1, wherein:
a thickness of the optoelectronic device is less than 5 mm.

13. The optoelectronic device of claim 1, wherein:
a thickness of the optoelectronic device is less than 3 mm.

14. The optoelectronic device of claim 1, the bio-compatible packaging comprising:
a back packaging layer; and
a front packaging layer, attached to the back packaging layer; wherein
the back packaging layer and the front packaging layer are configured
to house the optoelectronic assembly, and
to form a hermetically sealed packaging for the optoelectronic assembly.

15. The optoelectronic device of claim 14, wherein:
the back packaging layer and the front packaging layer are attached by at least one of laser-welding and metal-to-metal seals.

16. The optoelectronic device of claim 1, the bio-compatible packaging comprising:
a back packaging layer;
a middle packaging layer, attached to the back packaging layer; and
a front packaging layer, attached to the middle packaging layer; wherein
the back packaging layer, the middle packaging layer, and the front packaging layer are configured
to house the optoelectronic assembly, and
to form a hermetically sealed packaging for the optoelectronic assembly.

17. The optoelectronic device of claim 16, wherein:
the back packaging layer, the middle packaging layer, and the front packaging layer are attached by at least one of laser-welding and metal-to-metal seals.

18. The optoelectronic device of claim 1, wherein:
the biocompatible packaging is configured to house the electronic module, the optoelectronic module, and the power source in connected spaces.

19. The optoelectronic device of claim 1, wherein:
the biocompatible packaging is configured to house the electronic module, the optoelectronic module, and the power source in at least two spaces, sealed from each other.

20. The optoelectronic device of claim 1, comprising:
at least one of a desiccant, a getter, silica, calcium, a moisture-reducing agent, and a moisture capture material.

21. The optoelectronic device of claim 1, further comprising:
a soft outer packaging, configured
to round edges and sharp features of the bio-compatible packaging.

22. The optoelectronic device of claim 21, the soft outer packaging comprising:
at least one of a polymer, silicone, and AcrySof.

23. The optoelectronic device of claim 1, wherein:
the optoelectronic module provides an adjustable optical power in the range of 0-4 diopters; and
the soft outer packaging provides an optical power in the range of 6-30 diopters.

24. The optoelectronic device of claim 1, wherein:
the biocompatible packaging includes at least one sealed feedthrough; and
the soft packaging material includes at least one external electrode, electrically coupled to the feedthrough and to external electronics, wherein:
the electrode and the feedthrough form a signal route for the external electronics to signal the optoelectronic assembly.

25. The optoelectronic device of claim 1, the electronic interconnect comprising:
a bottom metal layer;
a bottom insulating layer, on the bottom metal layer to insulate the bottom metal layer;
an interconnect metal layer, on the bottom insulating layer, patterned to form electrical connections between feedthrough contacts;
a patterned top insulating layer, on the interconnect metal layer to insulate the interconnect metal layer, and patterned to form feedthrough contacts; and
a top metal layer, on the top insulating layer, configured
to complete a hermetic seal of the electronic interconnect together with the bottom metal layer and a side seal structure, and
to accommodate the feedthrough contacts.
